(19) United States
(12) Patent Application Publication
(10) Pub. No.: US 2021/0097883 A1
(43) Pub. Date: Apr. 1, 2021

(54) TAROT-BASED SYSTEM AND METHOD FOR DELIVERING A MENTAL HEALTH RESOURCE

(71) Applicant: INAG, Inc., Minden, NV (US)

(72) Inventor: Kenneth Alan Scott, (US)

(21) Appl. No.: 17/033,158

(22) Filed: Sep. 25, 2020

Related U.S. Application Data
(60) Provisional application No. 62/906,107, filed on Sep. 26, 2019.

(51) Int. Cl. G09B 19/00; G09B 5/02; G16H 20/70; G16H 10/60; G06Q 50/00; G06N 5/02

(52) U.S. Cl. CPC G09B 19/00; G09B 5/02; G16H 20/70; A63F 9/06; G06Q 50/01; G06N 5/02; G16H 10/60

(57) ABSTRACT

A fortune telling system using artificial intelligence and other resources is provided to direct specific helpful psychological and other resources to users through multiple methods. A method for delivering a mental health resource to a person includes conducting a Tarot reading regarding a situation by pulling a combination of Tarot cards from a Tarot card deck; and receiving, by a computer processor, information regarding the combination of Tarot cards. The method also includes determining, by a first artificial intelligence (AI) algorithm, an emotional response likely to result from the combination of Tarot cards; and generating, by a second AI algorithm, a message based upon the emotional response likely to result from the combination of Tarot cards. The message is constructed to provide insight or comment to the person regarding the situation.

### CROSS REFERENCE TO RELATED APPLICATION

This application claims priority to U.S. Provisional Application Ser. No. 62/906,107 entitled TAROT BASED GAME AND GAMING SYSTEM, filed Sep. 26, 2019, which is incorporated herein by reference in its entirety.

### TECHNICAL FIELD

The present disclosure relates generally to a method for delivering a mental health resource to a person. More specifically, the present disclosure relates to delivering a mental health resource in association with a Tarot-based game.

### BACKGROUND OF THE DISCLOSURE

Women spend about 75% of the hundreds of millions of dollars expended each year on psychics, Tarot readings, and other forms of mystic divination online and in-person, according to the New York Times. Tarot is booming, becoming increasingly popular with millennials.

Whether it's violence towards themselves, such as suicide, or violence towards others, such as shootings at school, we see it often in the news. Teens are turning violent, crying out for help. The mental health/mental illness of adolescents is becoming of major importance in the USA.

Suicide is the second leading cause of death for teens between the ages of 15 to 24. Suicide accounts for 18 percent of deaths among people of this age group. A study recently published in the Journal of Abnormal Psychology finds the percentage of U.S. teens and young adults reporting mental distress, depression and suicidal thoughts and actions has risen significantly over the past decade. While these problems have also increased among adults 26 and older, the increase was not nearly as large as among younger people. Over one-third of teens between the 9th and 12th grade show symptoms of major depression, according to the Childtrends, a non-profit research organization that analyzed statistics gathered from CDC national data sets. The same study showed that 39% of girls have reported feeling sad, depressed and hopeless, almost double the number of boys who report feeling the same way the same way (21-percent). They found the rate of individuals reporting symptoms consistent with major depression over the past year increased 52 percent in teens and 63 percent in young adults/millennials over the decade. Girls were more vulnerable than boys. In 2017 one out of every five teenage girls had experienced major depression in the last year. Psychological distress, described as “feeling nervous, hopeless or that everything in life is difficult” rose by 71 percent among people aged 18 to 25. Suicidal thoughts, plans and attempts also increased. Death from suicide increased by 56% among 18 and 19-year-olds between 2008 and 2017. Most people who commit suicide feel like it is their only way out of a helpless situation.

However, there are many resources for people with suicidal thoughts. With therapy and medication, many find that life is worth living. In fact, most people who attempt suicide say they regret it. Interviews with 29 people who survived a suicide attempt jumping off the Golden Gate Bridge say they regretted the decision the moment they jumped. Sadly, only 30% of depressed teens are being treated for their psychological illness.

The rise in smartphone and social media use correlate as significant factors. By 2012, smartphones had become widespread, and it's around that same time that social media began to dominate young people's lives. For example, in 2009 about half of high school seniors visited social media sites every day. That's now climbed to about 85 percent, with Instagram and Snapchat replacing Facebook as the preferred social media sites. It's not just the phone or social media itself. It's the amount of time teens and young adults spend with it which can be factored. Recent research has found the more time young adults spend, the greater the risk of depression.

According to Common Sense Media, teens spend an average of nine hours a day online (paywall), compared to about six hours for those aged eight to 12 and 50 minutes for kids between 0 and eight. That is a lot of time staring at a screen. Young adults are more worried about peer status and approval during pre-teen and teenage years. Social media exaggerates that process because it's so public, available, and highly visual. Social Media takes what happens in typical adolescent development and puts it on steroids. For example, in the past girls just went to school when they had bad hair, but now with social media there is suddenly a picture of you with bad hair. Everyone is now going to see it, comment on it, and make fun of them. These experiences resonate during adolescence and deeply affect teen's confidence and sense of self, leading to significant depression. Young adults are developmentally more worried about peer status and approval during teenage years. Social media exaggerates that process because it's so public, available, and highly visual. It takes what happens in typical adolescent development and puts it on steroids.

Researcher and clinical psychologist Steve Ilardi, with the University of Kansas tries to help anxious and depressed kids feel better. Ilardi developed a treatment approach, based in part on behavioral therapy, that gives young people the resources that help them make lifestyle changes. They encourage focusing on better diet and nutrition, exercise, exposure to sunlight and getting a good night's sleep, all of which have been shown to reduce depressive symptoms. “Kids buy into it,” says Ilardi, “when you lay it out for them and explain they can be empowered to make changes themselves that can make a big difference in how they feel, how your brain, mind and body functions.” He says behavior change can “help kids get unstuck from their perpetual sense of anxiety, stress and depression.”

Additionally, an astounding 22% of millennials surveyed say they have “no friends” and 27% say they have “no close friends”. However, according to the Pew Research Center, 72% of teens now play video games. This is the case among all young people: 72% of men ages 18 to 29 play video games, compared with 49% of women in the same age range.

Women however consistently prefer alternate styles of games. Women who often or sometimes play video games are more likely than men to play puzzle games (72% vs. 52%). Women are the overwhelming audience for Tarot and fortune-telling. Women are more able to be satisfied by emotional things than men. Fortune telling is purely emotional. There is no way to test it . . . it is designed to short circuit and overwhelm the emotionally dominant brain.

Teens frequently overshare and are willing to disclose personal information, with nearly seven out of ten teens receiving support through the internet during tough times. Girls are more likely to report having been supported, with nearly three-quarters (73%) of girls receiving support. The internet and Social Media connects teens to information.

Medical devices, including remote patient monitoring tools and consumer-focused wearables, will continue to advance, bringing real-time health data streaming into clinical environments. A lot of heart rate, glucose, and blood pressure monitors can now send information in real-time, which is a game-changing development. Now, you can collect the data without needing the person to set one-foot inside a clinic, which is revolutionary, because that information comes in immediately, and at a low cost-point. Physiological data including heart rate variability, (HRV) analysis, parasympathetic nerve tone and salivary cortisol levels are frequently used to access and monitor levels of major depression.

According to Healthanalytics.com in a May 2018 article, combining electronic health record (EHR) data and results from a depression questionnaire can support a more predictive analytics model that more accurately predicts suicide risk. The study, conducted by the Mental Health Research Network and Kaiser Permanente researchers, combined questioner data with a variety of EHR data from individuals receiving care. The study found that suicide attempts and deaths in the highest 1% of predicted risk were 200 times more common than among the people in the bottom half of predicted risk. People with risk scores in the top 5% accounted for 43% of suicide attempts and 48% of suicide deaths.

Earlier assessment models and techniques, using fewer data points were less accurate. Traditional suicide risk assessment, using only questionnaires and/or clinical interviews are far less accurate. The conclusive results of the study showed that using electronic health record data combined with other tools can accurately identify people at high risk for suicide attempt and suicide death. It is therefore very important to find more accurate ways of predicting suicide risk, particularly since the risk appears to be increasing, especially among possibly more susceptible millennials.

### SUMMARY OF THE INVENTION

According to an aspect of the disclosure, a method for delivering a mental health resource to a person includes conducting a Tarot reading regarding a situation by pulling a combination of Tarot cards from a Tarot card deck; and receiving, by a computer processor, information regarding the combination of Tarot cards. The method also includes generating, by an artificial intelligence (AI) algorithm, a message constructed to provide insight or comment to the person regarding the situation; and transmitting the message, by the computer processor, to the person.

According to an aspect of the disclosure, a method for delivering a mental health resource to a person includes conducting a Tarot reading regarding a situation by pulling a combination of Tarot cards from a Tarot card deck; and receiving, by a computer processor, information regarding the combination of Tarot cards. The method also includes determining, by a first artificial intelligence (AI) algorithm, an emotional response likely to result from the combination of Tarot cards; and generating, by a second AI algorithm, a message based upon the emotional response likely to result from the combination of Tarot cards. The message is constructed to provide insight or comment to the person regarding the situation.

According to an aspect of the disclosure, a system for delivering a mental health resource to a person includes a user interface configured to present output data to the person and to receive input data from the person. The system also includes a computer processor configured to receive the input data regarding a situation from the person via the user interface and to cause the user interface to present the output data regarding a Tarot reading. The Tarot reading includes pulling a combination of Tarot cards from a Tarot card deck. The system also includes a first artificial intelligence (AI) algorithm configured to determine an emotional response likely to result from the combination of Tarot cards; and a second AI algorithm configured to generate a message based upon the emotional response likely to result from the combination of Tarot cards. The message is constructed to provide insight or comment to the person regarding the situation.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIG. 1 shows a Tarot card deck according to an aspect of the present disclosure;

FIG. 2 shows a first example spread according to an aspect of the disclosure;

FIG. 3 shows a first example spread according to an aspect of the present disclosure;

FIG. 4 is a block diagram showing components within a system according to some aspects of the present disclosure;

FIG. 5A is a flow chart listing steps in a method for delivering a mental health resource to a person; and

FIG. 5B is a continuation of the flow chart of FIG. 5A.

### DESCRIPTION OF THE ENABLING EMBODIMENTS

Referring to the Figures, wherein like numerals indicate corresponding parts throughout the several views, a system and method for delivering a mental health resource to a person disclosed.

The system and method of the present disclosure involves using traditional and non-traditional methods and means of mystic divination combined with artificial intelligence (AI) to deliver mental health resources and develop critical thinking skills of psychological benefit to players that would be far beyond what is normally associated with that or any particular form of game play or fortune telling on its own. The actual item being delivered, may comprise communication of content to a user, with such content including indicia, animation, information and/or audio signals. The delivery methods and systems may include communication with or to a user, which may be via multimedia, audio or display devices, such as those associated with a computerized platform such as a mobile device, smartphone, PC, arcade amusement device or electronic gaming machine.

The system and method of the present disclosure can provide for more accurate suicide risk prediction and management, combined with game play and game play intake questions to follow up with young adults at high-risk, and provide them with helpful materials, refer them to or provide intensive treatment and red flag them due to the indicated high-risk and other red flag symptoms such as withdrawal from activities or cancelled doctor or therapy visits.

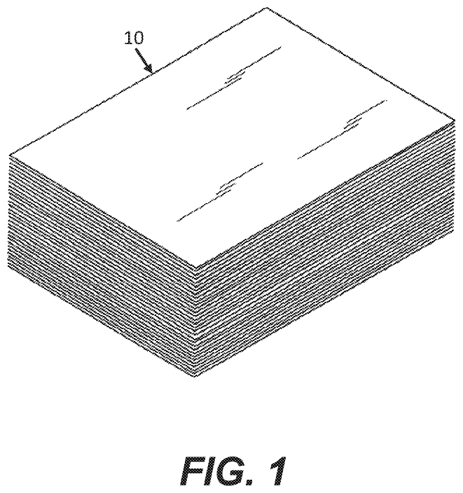

FIG. 1 shows a depiction of a Tarot card deck 10 in accordance with some aspects of the present disclosure. Unless otherwise defined, the term “Tarot cards” or “Tarot card deck” 10 includes any grouping of physical, virtual, or simulated cards that is identified and/or designed for providing Tarot readings. The term “Tarot cards” or “Tarot card deck” 10 may include any deck of physical, virtual, or simulated cards used or intended to be used for the purpose of mystic divination and/or fortune telling.

Use of Tarot game to Provide Comfort and Advice

In accordance with an aspect of the disclosure, a method is provided for using the fortune-telling medium and combinations of items within that medium to indicate to the AI to generate certain appropriate responses. For example, a particular combination of Tarot cards fall within a particular reading. The AI is programmed to recognize the content of those cards and identify and predict that that combination would engender a particular emotional response in a typical player. That recognition by the AI would then trigger a specific response to be generated and communicated to the player. That response could include an e-mail or other message to be sent or given to the player that supports the conclusion drawn with the intent of giving further insight or comment on the player's situation in the effort to give comfort or advice to that player and the situation.

### Spread Selection as Indicator

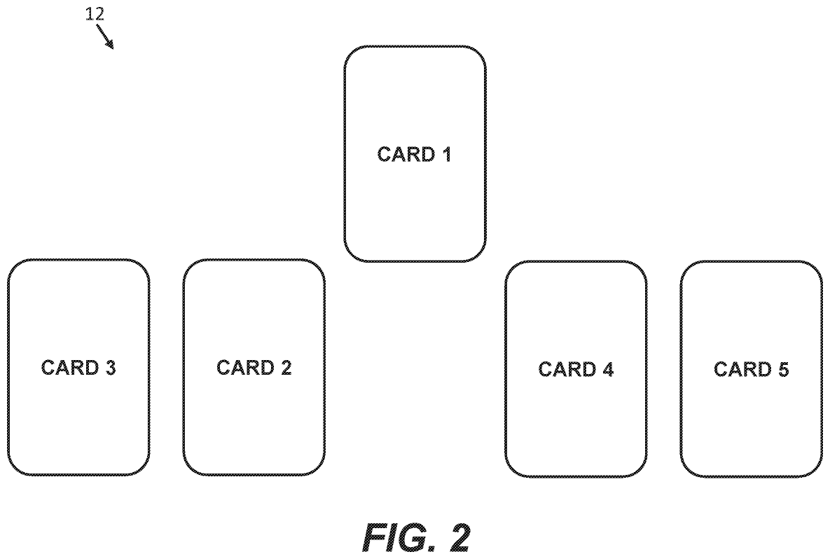

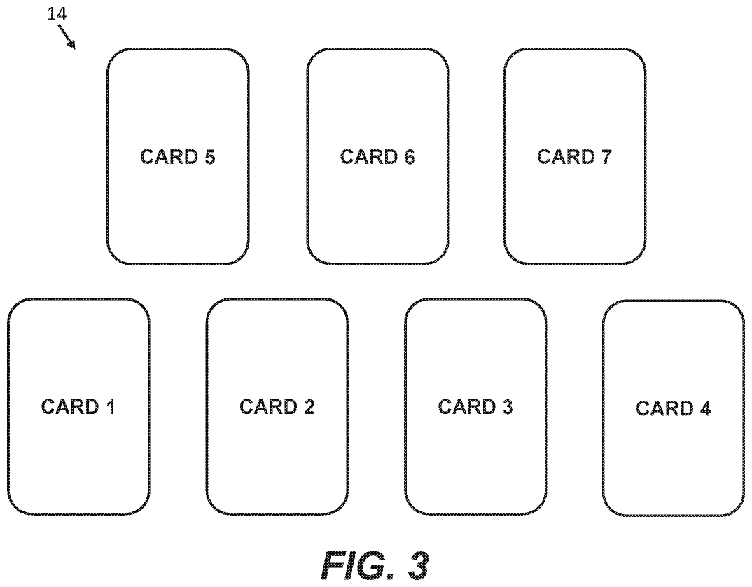

The next method involves the construction in Tarot that readings are divided amongst various patterns or “spreads” that have names regarding the areas to which the spreads relate. The player's selection of the type of spread is an immediate indicator of the main area(s) of concern. FIG. 2 shows a first example spread 12 having a particular arrangement with places for five cards, labeled CARD 1 through CARD 5, which are pulled from the Tarot deck 10. Each card place in the first example spread 12 represents a particular aspect of the reading. FIG. 3 shows a second example spread 14 having a different arrangement than that of the first example spread 12. The second example spread 14 includes places for seven cards, labeled CARD 1 through CARD 7, which are pulled from the Tarot deck 10. Each card place in the second example spread 14 represents a particular aspect of the reading.

In Tarot, spreads are categorized by the type of reading typical players desire and the number of cards per spread. For example, types of readings may be concerned with matters such as:
1) True love and its various forms like soul-mates, the search for love, number of children and the status of current relationships;
2) Wealth and financial concerns like career choices, forecasts about success and how to obtain it . . . “Will I be rich?” and “How can I advance in my career?”
3) Luck, happiness and fortune in the present and future . . . “Will I be happy?”
4) The When's of life . . . When will I find my dream job? When will I meet my soul-mate? When will my business take-off? When will I be happy?
5) Health . . . “Will I live a long and healthy life?” “When will I feel better?”
6) Friendship . . . “How can I make real friends?” “How can I find better friends?” and “How can I end a toxic relationship without bad feelings?” and;
7) Grief . . . “What can I do to overcome grief?” or “How can I make the person(s) I have lost proud?

In another example, the types of readings can be broken down by categories of spreads such as the following:

1. Spreads for Motivation

2. Tarot Spreads for Gratitude

3. Tarot Spreads for Self-Reflection

4. Spreads for Taking Action

5. Spreads for Manifestation

6. Spreads for Considering a Situation

7. Spreads for Timelines or Cycles

8. Spreads for Practicing Spirituality

9. Spreads for when you are in Conflict

10. Spreads for Social and/or Romantic Relationships

11. Spreads for Healing

12. Spreads for Overcoming Obstacles

13. Spreads for Money and Finance

14. Spreads for Career Decisions

15. Spreads for Achieving your goals

16. Spreads for When you need Advice

The frequency at which a player is selecting certain spreads will also be used as indicators of the player's emotional state. These selections identify consistent selection of spreads as indicators of stress, depression, grief, etc. Key questions are written for each tarot card spread “slot” which are answered by what card lands in that card slot. These two factors would also be interpolated into the response from the AI.

Music Selection for Additional Support and Emotional Stabilization

Depression causes changes in mood and loss of interest and pleasure. Music therapy, an intervention that typically involves regular sessions with a qualified music therapist, may help in improving mood through emotional expression.

### Intake Questions for Additional Information

As part of gameplay or as a follow-up to gameplay where a player visits a website or smartphone app to retrieve a recent reading, or to play various contests or receive bonuses, the player will be asked a variety of questions to assess their current mental state. PHQ's (Patient Health Questionnaires) can be easily re-written in a friendly, fun game play manner asking about mood, sleep habits, interests and more. These re-written questions actually regarding depression, happiness, general state of mind, relationships, etc., will be posed under the guise and in a way to make their next Tarot reading more accurate. Their answers will be combined with EHR data, and various forms of social media mining to enhance accuracy and further delineate their areas of concern and susceptibility to depression and possible other harmful behaviors.

### Social Media Mining

The next method uses the social media mining capabilities of AI to search a specific player's Facebook, Twitter, Instagram, Snapchat, e-mails (as possible and legal) and any other forms of social media usage to identify areas of concern or mental health of a player so that appropriate resources can be directed to address those needs. For example, if the player uses key phrases such as “stress,” “depressed,” “lonely,” “alone,” or other words or phrases that would indicate a state of depression or confusion, the AI would direct the player to various articles, TED talks, websites, help lines or other resources that would be helpful to that player's emotional state. Helpful resources can be refined by using genealogy and the detection of loss of family or friends. Red flag words like “suicide” could trigger numbers to or calls from preventative hotlines and/or free FaceTime chats to doctors, counselors or even emergency services. The AI could even send out guised alerts out to that player's contact list so that those contacts could reach out personally to that player. The AI could use tracking software to determine the player's location by geo-locating their device.

### Behavioral, Prescriptive and Predictive Analytics for Crisis Intervention

The system and method of the present disclosure uses behavioral, prescriptive and predictive analytics in combination with many other data points to assist players whose responses indicate that they are either in or heading toward crisis with the goal of helping them while they are still relatively mentally stable to develop the critical thinking skills required to help remove themselves from those negative thought cycles that could lead to harming themselves or spiral into a daily state of fighting for their lives.

### Behavioral Analytics

Behavioral analytics is an area of data analytics that focuses on providing insight into the actions of people, usually regarding online purchasing. Behavioral analytics is used in ecommerce, gaming, social media, and other applications to identify opportunities to optimize in order to realize specific business outcomes.

Behavioral analytics is based off hard data. It uses the volumes of raw data people use while they're on social media, in gaming applications, marketing, retail sites, or applications. This data is collected and analyzed, and then used as the basis of making certain decisions, including how to determine future trends or business activity, including ad placement.

However, there is a lot of ambiguity about the nature of the insights that it yields. For example, online advertisers use behavioral analytics to help them tailor the right offer at the right time. This is often done utilizing the user's demographic data, any past search or social information, and a locational market to put the user into a bigger group, sometimes called a cohort or demographic. The user is then served with ads or offers that match the ads and offers that have the highest success rate with that group.

Behavioral analytics can support a number of different hypotheses, so the process of elimination comes from experimentation and evaluation. Businesses usually are looking to increase conversions, so if the change makes it worse, that hypothesis can be thrown out in favor of a different one or no change at all. Behavioral analytics are most often used to inform A/B testing where one variable is changed at a time. As behavioral analytics have deepened and the technology to test multiple changes in real time evolves, companies are getting much better at targeting customers.

The A-B split is a method of testing the effectiveness of marketing methods or media. Using A-B split marketing, a list of target names is split into two groups on a random basis, with one group designated as a control group and the other as a test or variation group. The objective of the A-B split is to determine which single variable is the most effective in improving response rates to a marketing campaign or achieving some other desired outcome. The A-B split is also referred to as “A/B testing,” “bucket tests” or “split-run testing.”

### Types of Behavioral Analytics

As is to be expected, behavioral analytics is usually employed for the purpose of driving sales, either via ad placement or suggested products.

E-commerce and retail: This type helps make product recommendations and future sales trends based on consumers' current tastes. Online gambling: This helps predict trends in usage and preferences for future offerings. As gaming companies move away from a packaged product, they use behavioral analytics to target their garners on specific, in-game upsells. Application development: Businesses can figure out how people use an app to forecast future trends. As with online gaming analytics, companies will offer upgrades within the app based on behavioral patterns. Security: This type of analytics helps detect compromised information by finding unusual activity, and is employed by both government agencies and private companies throughout the world.

### Criticism of Behavioral Analytics

Amazon offers a personalized homepage based on demographics, past purchases, search queries, and products viewed using behavioral analytics, and each product page shows you what people just like you did after viewing that page. This data trove is the real power behind Amazon. Starting in 2015, Amazon was among tech companies like Google in releasing in-home voice products that should become a wealth of behavioral analytics regarding off-line life just, as your actions on their sites are a source of data for your online life. Some people view this as intrusive and overly informative to both the data companies and government, but they are de facto agreeing to the terms when they purchase the item.

### Key Takeaways

Behavioral analytics is used to track users' preferences and offer or direct that user to targeted content. Mostly it is used to drive potential customers to specific products or advertisements. Some feel that the systems put in place to collect data are harmful and intrusive, worrying that everything they do is tracked and monitored.

### Prescriptive Analytics

Prescriptive analytics is a type of data analytics—the use of technology to help businesses make better decisions through the analysis of raw data. Specifically, prescriptive analytics factors information about possible situations or scenarios, available resources, past performance, and current performance, and suggests a course of action or strategy. It can be used to make decisions on any time horizon, from immediate to long term. The opposite of prescriptive analytics is descriptive analytics, which examines decisions and outcomes after the fact.

### How Prescriptive Analytics Works

Prescriptive analytics relies on artificial intelligence techniques, such as machine learning—the ability of a computer program, without additional human input, to understand and advance from the data it acquires, adapting all the while. Machine learning makes it possible to process the tremendous amount of data available today. As new or additional data becomes available, computer programs adjust automatically to make use of it, in a process that is much faster and more comprehensive than human capabilities could manage.

Prescriptive analytics works with another type of data analytics, predictive analytics, which involves the use of statistics and modeling to determine future performance, based on current and historical data. However, it goes further: Using the predictive analytics' estimation of what is likely to happen, it recommends what future course to take.

Prescriptive analytics is not foolproof, however. It is only effective if organizations know what questions to ask and how to react to the answers. If the input assumptions are invalid, the output results will not be accurate.

When used effectively, however, prescriptive analytics can help organizations make decisions based on highly analyzed facts rather than jump to under-informed conclusions based on instinct. Prescriptive analytics can simulate the probability of various outcomes and show the probability of each, helping organizations to better understand the level of risk and uncertainty they face than they could be relying on averages. Organizations can gain a better understanding of the likelihood of worst-case scenarios and plan accordingly.

### Key Takeaways

Prescriptive analytics makes use of machine learning to help businesses decide a course of action based on a computer program's predictions. Prescriptive analytics works with predictive analytics, which uses data to determine near-term outcomes. When used effectively, prescriptive analytics can help organizations make decisions based on facts and probability-weighted projections, rather than jump to under-informed conclusions based on instinct.

### Examples of Prescriptive Analytics

Numerous types of data-intensive businesses and government agencies can benefit from using prescriptive analytics, including those in the financial services and health care sectors, where the cost of human error is high.

Prescriptive analytics could be used to evaluate whether a local fire department should require residents to evacuate a particular area when a wildfire is burning nearby. It could also be used to predict whether an article on a particular topic will be popular with readers based on data about searches and social shares for related topics. Another use could be to adjust a worker training program in real time based on how the worker is responding to each lesson.

### Prescriptive Analytics for Hospitals and Clinics

Similarly, prescriptive analytics can be used by hospitals and clinics to improve the outcomes for patients. It puts healthcare data in context to evaluate the cost-effectiveness of various procedures and treatments and to evaluate official clinical methods. It can also be used to analyze which hospital patients have the highest risk of re-admission so that healthcare providers can do more, via patient education and doctor follow-up to stave off constant returns to the hospital or emergency room.

### Predictive Analytics

Predictive analytics describe the use of statistics and modeling to determine future performance based on current and historical data. Predictive analytics look at patterns in data to determine if those patterns are likely to emerge again, which allows businesses and investors to adjust where they use their resources to take advantage of possible future events.

### Key Takeaways

Predictive analytics is the use of statistics and modeling techniques to determine future performance. It is used as a decision-making tool in a variety of industries and disciplines, such as insurance and marketing. Predictive analytics and machine learning are often confused with each other but they are different disciplines.

### Understanding Predictive Analytics

There are several types of predictive analytics methods available. For example, data mining involves the analysis of large tranches of data to detect patterns from it. Text analysis does the same, except for large blocks of text.

Predictive models look at past data to determine the likelihood of certain future outcomes, while descriptive models look at past data to determine how a group may respond to a set of variables. Predictive analytics is a decision-making tool in a variety of industries. For example, insurance companies examine policy applicants to determine the likelihood of having to pay out for a future claim based on the current risk pool of similar policyholders, as well as past events that have resulted in payouts. Marketers look at how consumers have reacted to the overall economy when planning on a new campaign, and can use shifts in demographics to determine if the current mix of products will entice consumers to make a purchase. Active traders look at a variety of metrics based on past events when deciding whether to buy or sell a security. Moving averages, bands and break points are based on historical data, and are used to forecast future price movements.

### Common Misconceptions of Predictive Analytics

A common misconception is that predictive analytics and machine learning are the same things. At its core, predictive analytics includes a series of statistical techniques (including machine learning, predictive modeling, and data mining) and uses statistics (both historical and current) to estimate, or predict, future outcomes. Predictive analytics help us to understand possible future occurrences by analyzing the past. Whereas machine learning, on the other hand, is a subfield of computer science that, as per the 1959 definition by Arthur Samuel—an American pioneer in the field of computer gaming and artificial intelligence which gives “computers the ability to learn without being explicitly programmed.”

The most common predictive models include decision trees, regressions (linear and logistic) and neural networks—which is the emerging field of deep learning methods and technologies.

### Example of Predictive Analytics

Forecasting is an essential task in manufacturing because it ensures optimal utilization of resources in a supply chain. Critical spokes of the supply chain wheel, whether it is inventory management or shop floor, require accurate forecasts for functioning. Predictive modeling is often used to clean and optimize the quality of data used for such forecasts. Modeling ensures that more data can be ingested by the system, including from customer-facing operations, to ensure a more accurate forecast.”

### Using Behavioral, Prescriptive & Predictive Analytics for Crisis Intervention.

The system and method of the present disclosure uses its social media mining, intake questions, response to resources offered, and monitoring of players' spread selections and frequency of play functions combined with behavioral, prescriptive and predictive analytical functions to predict the type of resources and other help that would most likely be helpful to a player in need to intervene in a crisis that the player may be experiencing.

Many people who have survived a suicide attempt say that they did it because they mistakenly felt there was no other solution to a problem they were experiencing. At the time, they couldn't see another way out, but in truth, they didn't really want to die. Additional Goals

An additional goal of the system and method of the present disclosure is to encourage and provide people with skills to develop authentic friends and social relationships, so he or she can experience the relief of being heard, understood and validated by friends and loved ones, and build genuine connections.

Another important factor that can be encouraged and taught is the adoption of healthy habits. Making healthy lifestyle choices can do wonders for the mood. Things like eating right, regular exercise, sleeping regularly and avoiding alcohol and “recreational” drugs have been shown to make a huge difference in depression. Physical activity can be as effective as medications or therapy for depression. The brain is drawn to fun, so identifying interests and encouraging individuals to become active in preferred sports, riding their bikes, walking the family dog, or taking dance classes can really help.

### Communicating as a Member of a Targeted Demographic

In the next method, the AI can be programmed to emulate a particular type of demographic so that the player receives feedback or messaging from a character that is expected by or most relatable to that player. For example, if the player is a young female, she may relate best to another young female. Therefore, in most instances, an AI programmed to respond in the same speech and writing patterns or slang as a young female will be most relatable to that young female player.

### Critical Thinking Skills Development

The AI can be programmed to help the player develop critical thinking skills and coping mechanisms by delivering such advice and guidance in the most relatable format by adopting the characteristics of general demographic of the player or players. If the AI speaks or writes or otherwise communicates those critical thinking skills and coping mechanisms to a player in the voice and/or style of a person having an identical or a similar demographic of that player, said skills and mechanisms will be more readily directed and accepted.

Additionally, critical thinking is often taught using images, such as tarot card imagery. Tarot cards can also be used and function as standard Rorschach inkblots stimulating the imagination and making important emotional associations. Tarot card images coupled with properly phrased psychologically probing questions (verbalized within the readings, e-mails, card slots, and posed by the AI fortune-teller) bring forth information buried deep within the unconscious, sparking the imagination to draw upon deep hidden levels of consciousness. When used with modern counseling techniques and questions, tarot card imagery is a powerful tool to build inner resources while uncovering underlying anxieties and concerns.

### Facial Expression Identification

The next method uses a camera that records the emotional expression of a player during a reading or a specific part of a reading so that the AI system could read and understand that emotional expression. When a particular emotion is detected, the AI would send or otherwise generate a message that supports, consoles, counsels or advises the player's emotional status as related to the tarot cards displayed. The AI recognizes the facial expression and interacts with the player's emotional state and relates supportive information on an appropriate level.

### Imagery Selected to Impact Player Reaction

A 2009 study by the Department of Psychiatry and Stress Research Institute in cooperation with the Seoul Paik Hospital in Seoul, Korea that psychotherapeutic treatment applied in a forest produced increased psychological changes and remission of Major Depressive Disorder (MDD).

A study conducted therapy performed on patients in three different settings, a hospital, a control environment and a forest. The remission rate of the forest group was significantly higher (61%) than both the hospital group and the control group. After four sessions, HRV (heart rate variability) parasympathetic nerve tone were improved along with salivary cortisol levels being significantly decreased.

Beautiful, 3D immersive backgrounds using some of the most beautiful forests and natural settings in the world will therefore be created and utilized. Additional imagery can also be designed by AI to illicit particular psychological responses from the player. For example, an individual Tarot card can contain imagery can be specifically designed to improve the players' emotional reaction to that card.

### Stylized Writing to Impact Reactions

The method of delivering a fortune can also be constructed in cooperation with clinical psychologists and AI to deliver psychological benefits. The Tarot readings themselves can be written and delivered in an optimistic and hopeful manner so that optimism and hope are generated within the player. In the same way, situational readings that require caution and awareness of the player's surroundings can be written so that introspection and deep thought are generated in the player's mind so that careful and thoughtful decisions are made.

### Stylized Art, Animation & Music

The method of delivering a fortune can also be influenced by the style of artwork, animations and music used in the device. The style of imagery in the artwork can be presented with humourism or in a romanticized fashion so that the environments and characters' attractiveness is highlighted or exaggerated much in the way that the cover art of romance novels present the women as ravishing and the males are made to be extremely muscular and handsome. These animations can be presented in manners including holograms, 3D immersive and 4D effects technology to draw the viewer into the fortune teller's world more deeply than traditional animations would. Backgrounds, the fortune teller's wardrobe, the setting and other supportive characters such as cats, owls or other known familiars will add to the scene's authenticity and effectiveness while improving the visual experience for the player and immersing them more deeply into the fortune teller's world. The choice of music can also influence the player's experience such as music therapy is applied in treatment of depression.

### Gaming Applications

A Tarot deck 10 consists of seventy-eight cards with four suits and 22 Major Arcana cards. The Tarot cards are numbered Ace or one (1) through ten (10) with four face cards named the Princess or Page, the Prince or Knight, the Queen and the King. Each suit contains these cards. The suits are named Coins or Pentacles, Swords, Cups, and Wands. The numbering is represented by a number of pips or the number of representations of the symbol for that suit on a particular card. For example, the three (3) of Coins has 3 coins on the card. Each of the cards has a meaning dependent on the type of pattern or spread into which it is dealt. All of these cards are referred to as Minor Arcana cards.

Each suit has a general to specific meaning as well. Coins relate to health, wealth, material possessions and the realization of ideas and material gain. Swords are symbolic of battles within, challenge, physical and moral conflict, action and change. Cups relate to positive and negative emotions, love, relationships, romance, happiness, intuition, psychic abilities and spiritual connections. Wands are symbolic of passion, creativity, energy, career and ideas.

The Major Arcana cards have the following numerical value in the popular Rider-Waite deck and are named the Fool (0), the Magician (1), the High Priestess (2), the Empress (3), the Emperor (4), the Hierophant (5), the Lovers (6), the Chariot (7), Strength (8), the Hermit (9), Wheel of Fortune (10), Justice (11), the Hanged Man (12), Death (13), Temperance (14), the Devil (15), the Tower (16), the Star (17), the Moon (18), the Sun (19), Judgement (20), and the World (21).

The Fool is the main character of the Major Arcana and represents ourselves and our progression through life. As the Fool travels, he makes his journey through each of the cards, meeting the 21 archetypes and accepting them as different aspects of himself The archetypes are new teachers and he learns new life lessons along the way, and eventually reaching the completion of his journey with the World card. This is known as the Fool's Journey and is a helpful way of understanding the story line of the Major Arcana Tarot card meanings.

The Major Arcana Tarot cards represent the life lessons, karmic influences and the big archetypal themes that are influencing the players' lives and their souls' journeys towards enlightenment. The Major Arcana card meanings are deep and complex. These Tarot cards represent the structure of human consciousness and are believed to hold the keys to life lessons.

The gaming applications of the Tarot deck 10 could be varied or combined in a multitude of methods. The numerical value assigned to the Major Arcana cards could be calculated by their numerical value as trump cards and payouts could correlate to those values. Similarly, particular characters from the Major Arcana cards or the Major Arcana card itself or a grouping of Major Arcana cards could be specially designated for particular awards.

For example, the triggering outcome for a payout or bonus could be receiving some or only Major Arcana cards during gameplay or a reading. If a player received all Major Arcana cards in her reading (a Major Arcana flush), the payout could be based on the numerical value assigned to each Major Arcana card. If the player received the World (21), Judgement (20), the Sun (19), the Moon (18) and the Star (17) in a five (5) card reading, those points would total ninety-five (95) according to the Rider-Wait list above. The payment could be ninety-five (95) total points or an amount multiplied by that point total.

Awards could also be designated by suit. As the suit of Coins relates directly to material wealth, that suit would be the most logical choice. However, a justification for using any particular suit could be constructed and employed.

In a gaming application, a player could be rewarded by the number of Coins cards that the player receives during gameplay or a Tarot reading to build a Coins card flush for example. Also, as each Minor Arcana card is designated by a number of pips or representations of a particular suit, the player could be rewarded by an accounting of those number of pips or representations of that suit on a particular card.

It should be understood that a “gaming application” as used herein can refer to amusement games, non-wagering games, wagering games, free to play games or “freemium” games, virtual currency-based games, cryptocurrency-based games, as well as other promotional or electronic credit based games. For example, the system and method of the present disclosure can be embodied as a gaming application or device for amusement purposes only, wherein the Tarot reading is provided for entertainment, with or without an award, whether monetary or otherwise. Thus, embodiments of the system and method of the present disclosure include amusement devices configured to communicate content, consisting of advice, counseling or guidance, or other content that may be of the type resulting from a Tarot reading, without an award of any kind, even if payment is received from the user to actuate the device and receive such content.

As no current gambling or amusement game is in any way based on a Tarot deck 10, any of these structures would be unique. As the imagery in a particular Tarot deck 10 is unique, the use of a particular game could be viewed as having a distinctive deck 10 for an invented game. The gaming application will also be the first and only fortune-telling machine that can predict the player will become a millionaire and make it happen on the spot.

In some embodiments, a communication with a user responsive to certain actuating activity, such as, receiving a payment or wager, is based on the application of any or all of the above analytical methodologies, that is, behavior, prescriptive and predictive, either with or without interaction with the user, thus delivering content of a compelling nature in the form of a game. For example, the content delivered may be a multimedia display of a prediction of the user's fortune.

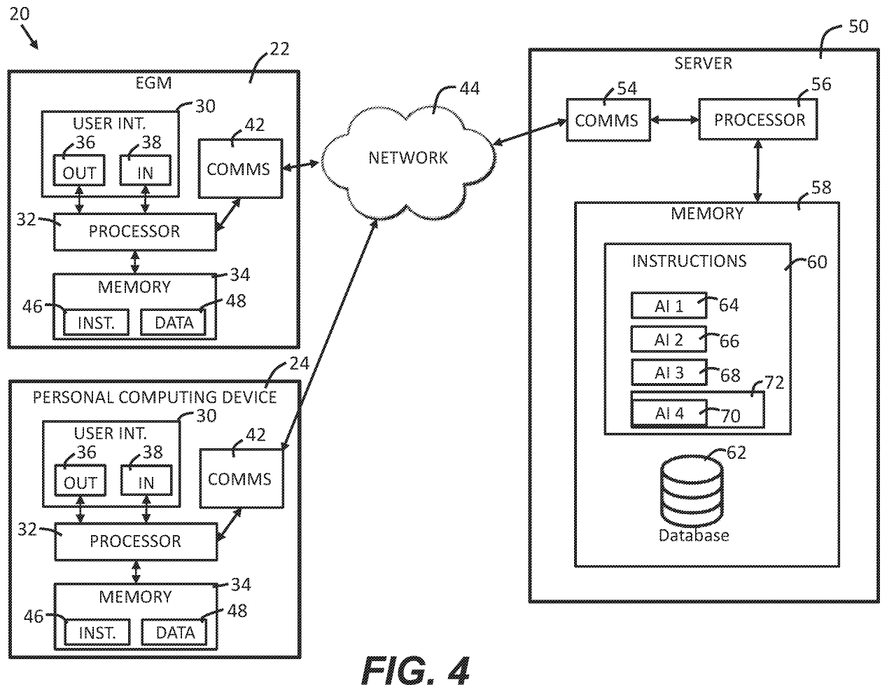

FIG. 4 is a block diagram showing components within a system 20 according to aspects of the present disclosure. The system 20 includes an electronic gaming machine (EGM) 22, and a personal computing device 24. These are merely examples, and the system 20 may contain any number of EGMs 22 and/or any number of personal computing devices 24. The personal computing device 24 or personal computing devices 24 may take the form of a laptop or desktop personal computer, a set-top box, a smartphone, a personal digital assistant, or another device. These are merely examples, and the personal computing device 24 may take another physical form. The personal computing device 24 includes similar or identical components to the EGM 22, so only the EGM 22 will be described here. The EGM 22 may take the form of a kiosk or a video gaming machine, such as a tabletop or a stand-alone casino gaming machine.

As illustrated in the example embodiment shown in the block diagram of FIG. 4, the EGM 22 includes a user interface 30, and a first processor 32 coupled to a first machine-readable storage memory 34. The user interface 30 includes an output device 36 configured to present output data to a user, and an input device 38 configured to receive input data from the user. The output device 36 may include a video display, such as a display screen, a projected display, or a virtual-reality (VR) or augmented reality (AR) image. Alternatively or additionally, the output device 36 may include audio output, such as one or more speakers providing the output in the form of audible signals. The output device 36 may include verbal communications, which may be computer generated, music, and/or other sound effects. The input device 38 may include a touch-screen, a keyboard, mouse, trackpad, trackball, gesture input. Alternatively or additionally, the input device 38 may include hardware and/or software to respond to verbal commands. The output device 36 may be combined with the input device 38, for example, as a touch screen.

As also shown in FIG. 4, the EGM 22 includes a first communications interface 42 configured to transmit and to receive data to/from a server 50 via a network 44. The first communications interface 42 may include a wired or a wireless interface, such as, for example, a Universal Serial Bus (USB) or Ethernet interface, or a Wi-Fi, Zigbee, or cellular data radio. The network 44 may include one or more wired and/or wireless segments, which may include, for example, Wi-Fi, Zigbee, Ethernet, infrared, etc.

The first machine-readable storage memory 34 may include one or more of a RAM memory, a ROM memory, flash, or DRAM and may include magnetic, optical, semiconductor, or another type of machine-readable storage. The EGM 22 also includes first instructions 46 stored in the first machine-readable storage memory 34 for directing the first processor 32 to cause the output device 36 to present particular output data to the user, and to cause the first processor 32 to receive feedback from the user via the input device 38 and to store data in a first data storage region 48 of the first storage memory 34 and to transmit the data to the server 50. The first instructions 46 may include compiled or interpreted data instructions that cause the first processor 32 to perform operations to enable the system 20 to function.

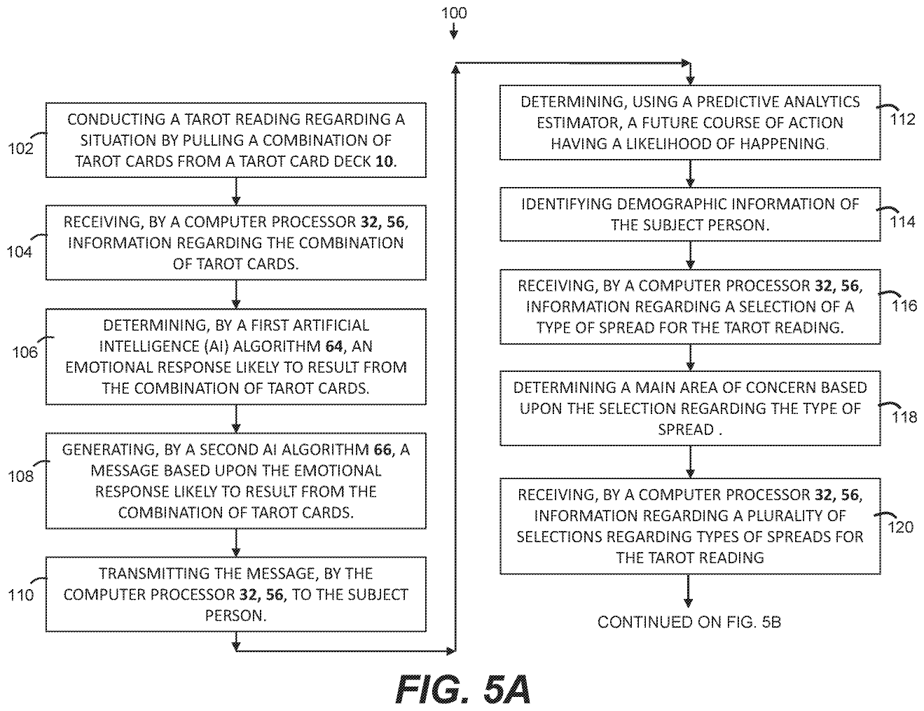

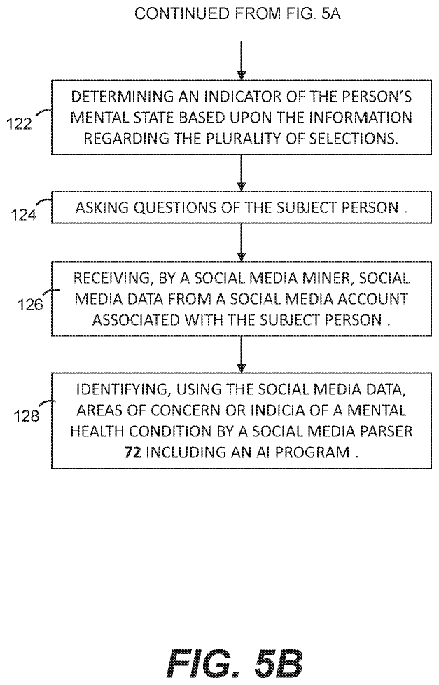

The server 50 includes a second communications interface 54 for communicating with the EGM 22 and/or for communicating with the personal computing device 24. The second communications interface 54 may include one or more wired and/or wireless interfaces, which may be the same type or a different type as the first communications interface 42. The server 50 also includes a second processor 56 and a second machine-readable storage memory 58 including second instructions 60 and a second data storage region 62 for storing data. The second data storage region 62 may be organized as a database, as shown on FIG. 5\4. Alternatively or additionally, data may be stored on an external database that is outside of the second machine-readable storage memory 58 of the server 50. For example, the data may be hosted on a dedicated database.

The second instructions 62 may be configured to cause the second processor 56 to store and analyze the data. In some embodiments, and as shown in FIG. 4, the second instructions 62 includes a first artificial intelligence (AI) algorithm 64, a second AI algorithm 66, a third AI algorithm 68, and a fourth AI algorithm 70. Specifically, the first AI algorithm 64 is configured to determine an emotional response likely to result from a particular combination of Tarot cards. The second AI algorithm 66 is configured to generate a message based upon the emotional response likely to result from the combination of Tarot cards. The message may be constructed to provide insight or comment to the user regarding a particular situation. Alternatively, the message may be constructed and intended for a person other than the subject person, such as a friend, family member, teacher, counselor, etc. Such a third-party message may include instructions and/or guidance for the person other than the user to provide help to the subject person regarding the particular situation.

The third AI algorithm 68 is used as a predictive analytics estimator to determine a future course of action having a particular likelihood of happening. The fourth AI algorithm 70 is part of a social media parser 72 and is used to identify, using the social media data, areas of concern or indicia of a mental health condition. The social media parser 72 may include other components, such as a scraper (not shown in the FIGs) to locate and download the social media via the Internet.

The AI algorithms 64, 66, 68, 70 may also be called AI programs, and may be embodied hardware and/or software. In some embodiments, the AI algorithms 64, 66, 68, 70 may be implemented using the same or shared or common hardware and/or software components. In some embodiments, one or more of the AI algorithms 64, 66, 68, 70 may include machine learning (ML) components. In some embodiments, and as shown in FIG. 4, each of the AI algorithms 64, 66, 68 is located within the second instructions 60 of the server 50. Alternatively, one or more of the AI algorithms 64, 66, 68 may be located elsewhere, such as within the first instructions 46 in the EGM 22 or the personal computing device 24.

A method 100 for delivering a mental health resource to a subject person is shown in the flow chart of FIGS. 5A-5B. The method 100 includes conducting a Tarot reading regarding a situation by pulling a combination of Tarot cards from a Tarot card deck 10 at step 102.

The method 100 continues with receiving, by a computer processor 32, 56, information regarding the combination of Tarot cards at step 104. This step 104 may include receiving an input information regarding physical cards that were pulled in step 102. Alternatively or additionally, this step 104 may include receiving information regarding virtual cards that were drawn, for example, where the Tarot reading is performed via computerized interface, such as on an EGM 22 or on a personal computing device 24.

The method 100 continues with determining, by a first artificial intelligence (AI) algorithm 64, an emotional response likely to result from the combination of Tarot cards at step 106.

The method 100 continues with generating, by a second AI algorithm 66, a message based upon the emotional response likely to result from the combination of Tarot cards at step 108. The message may be constructed to provide insight or comment to the subject person receiving the Tarot reading regarding a situation that is a subject of the Tarot reading. In some embodiments, the 108 of the method 100 may include using electronic health record (EHR) data regarding the subject person, by the second AI algorithm 66, for generating the message.

The method 100 continues with transmitting the message, by the computer processor 32, 56, to the subject person at step 110. In some embodiments, transmitting the message at step 110 includes sending an electronic mail (e-mail) message to the subject. Alternatively or additionally, the message may be transmitted to the subject person using a user interface 30 of the EGM 22 or the personal computing device 24. In some embodiments, the message may include guidance regarding critical thinking skills or coping mechanisms. In some embodiments, the message may include directions for obtaining a resource for help with the situation or with an identified emotional state.

The method 100 also includes determining, using a predictive analytics estimator, a future course of action having a likelihood of happening at step 112. In some embodiments, the predictive analytics estimator includes a third AI algorithm 68. Generating the message at step 108 may include providing a recommendation regarding the future course of action. For example, if the predictive analytics estimator determines that there is a sufficiently high likelihood that the subject person may hurt themselves or someone else, the message may be generated with specific recommendations or resources to prevent the subject person from hurting themselves or someone else or to reduce the risk of the user hurting themselves or someone else.

The method 100 also includes identifying demographic information of the subject person at step 114. The demographic information may include age, gender, location This step may include input information solicited directly from the subject person or information from another source, such as a public or private record associated with the person, from social media data, or from information provided by a third party, such as an administrator of the system 20. Step 108 of generating the message may include preparing the message in a voice or style based upon the demographic information of the subject person. For example, the message may be tailored using particular slang or speech or writing patterns commonly used by people having demographic information similar or identical to the subject person.

The method 100 also includes receiving, by a computer processor 32, 56, information regarding a selection of a type of spread for the Tarot reading at step 116, and determining a main area of concern based upon the selection regarding the type of spread at step 118. The main area of concern may then be used as a factor used by the AI algorithm for generating the message.

The method 100 also includes receiving, by a computer processor 32, 56, information regarding a plurality of selections regarding types of spreads for the Tarot reading at step 120, and determining an indicator of the person's mental state based upon the information regarding the plurality of selections at step 122. Step 108 of the method 100 may include using the indicator of the person's mental state, by the second AI algorithm 66, for generating the message.

The method 100 also includes asking questions of the subject person at step 124. The questions being designed to assess a current mental state of the subject person. This step 124 may be performed using the user interface 30 of the EGM 22 or the personal computing device 24. In some embodiments, the questions may be asked of the subject person in-person or remotely by an avatar or a human, such as a clinician, a therapist, or a psychic medium (i.e. a fortune teller). Step 108 of the method 100 may include using the answers to the questions, by the second AI algorithm 66, for generating the message.

The method 100 also includes receiving, by a social media miner, social media data from a social media account associated with the subject person at step 126. The social media miner may include one or more scraper software applications to locate and download the social media data from the Internet and/or from another source, such as from data stored on a smartphone, computer, or other device used by the subject person. The method 100 also includes identifying, using the social media data, areas of concern or indicia of a mental health condition by a social media parser 72 including an AI program, such as the fourth AI algorithm 70, at step 128. Step 108 of the method 100 may include using the areas of concern or the indicia of a mental health condition, by the second AI algorithm 66, for generating the message.

According to an aspect of the disclosure, a system 20 for delivering a mental health resource to a subject person is provided. The system 20 includes a user interface 30 configured to present output data to the subject person and to receive input data from the person. The system 20 also includes a computer processor 32, 56 configured to receive the input data regarding a situation from the subject person via the user interface 30 and to cause the user interface 30 to present the output data regarding a Tarot reading, the Tarot reading including pulling a combination of Tarot cards from a Tarot card deck 10.

The system 20 includes a first artificial intelligence (AI) algorithm 64 configured to determine an emotional response likely to result from the combination of Tarot cards. The system 20 also includes a second AI algorithm 66 configured to generate a message based upon the emotional response likely to result from the combination of Tarot cards. The message is constructed to provide insight or comment to the subject person regarding the situation.

In some embodiments, the user interface 30 is provided by an electronic gaming machine (EGM) 22. In some embodiments, the user interface 30 is provided by a personal computing device 24.

In some embodiments, at least one of the first AI algorithm 64 and/or the second AI algorithm 66 is provided as a computer program running on a server 50 that is located remote from the user interface 30 and in communication with the user interface 30 over a digital computer network 44.

In some embodiments, the system 20 also includes a predictive analytics estimator configured to determine a future course of action having a likelihood of happening. The second AI algorithm 66 may be configured to generate the message by providing a recommendation regarding the future course of action.

In some embodiments, the message includes guidance regarding critical thinking skills or coping mechanisms, or wherein the message includes directions for obtaining a resource for help with the situation or with an identified emotional state. In some embodiments, the message is transmitted as an electronic mail (e-mail) message to the subject person.

In some embodiments, the user interface is configured to ask questions designed to assess a current mental state of the subject person. The second AI algorithm 66 may be configured to use answers to the questions for generating the message.

The following description provides systems and methods for implementing features described above in gaming applications. The gaming applications may be implemented in accordance or in conjunction with one or more of a variety of different types of gaming systems, such as those described herein, including computer-based platforms which may be specially configured for the provision of wagering games, such as electronic gaming machines, or other devices which are not specially configured for the provision of wagering games, such as a smartphone, but which can be enabled as a platform through which such gaming applications including the game features of the systems and methods of the present disclosure can be made accessible. Embodiments of the invention therefore contemplate a variety of different gaming systems in and through which gaming applications of the present disclosure may be employed, each gaming system and/or method having one or more of a plurality of different features, attributes, or characteristics as disclosed herein.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

In some embodiments, the system may include data collection modules using machine learning to enable derivation-based learning outcomes from computers without the need to program them. The system may, therefore, learn from and make decisions on a set of data, by making data-driven predictions and adapting according to the set of data. In embodiments, machine learning may involve performing a plurality of machine learning tasks by machine learning systems, such as supervised learning, unsupervised learning, and reinforcement learning. Supervised learning may include presenting a set of example inputs and desired outputs to the machine learning systems. Unsupervised learning may include the learning algorithm itself structuring its input by methods such as pattern detection and/or feature learning. Reinforcement learning may include the machine learning systems performing in a dynamic environment and then providing feedback about correct and incorrect decisions. In examples, machine learning may include a plurality of other tasks based on an output of the machine learning system. In examples, the tasks may also be classified as machine learning problems such as classification, regression, clustering, density estimation, dimensionality reduction, anomaly detection, and the like. In examples, machine learning may include a plurality of mathematical and statistical techniques. In examples, the many types of machine learning algorithms may include decision tree based learning, association rule learning, deep learning, artificial neural networks, genetic learning algorithms, inductive logic programming, support vector machines (SVMs), Bayesian network, reinforcement learning, representation learning, rule-based machine learning, sparse dictionary learning, similarity and metric learning, learning classifier systems (LCS), logistic regression, random forest, K-Means, gradient boost and adaboost, K-nearest neighbors (KNN), a priori algorithms, and the like. In embodiments, certain machine learning algorithms may be used (such as genetic algorithms defined for solving both constrained and unconstrained optimization problems that may be based on natural selection, the process that drives biological evolution). By way of this example, genetic algorithms may be deployed to solve a variety of optimization problems that are not well suited for standard optimization algorithms, including problems in which the objective functions are discontinuous, not differentiable, stochastic, or highly nonlinear. In an example, the genetic algorithm may be used to address problems of mixed integer programming, where some components restricted to being integer-valued. Genetic algorithms and machine learning techniques and systems may be used in computational intelligence systems, computer vision, Natural Language Processing (NLP), recommender systems, reinforcement learning, building graphical models, and the like. By way of this example, the machine learning systems may be used to perform intelligent computing based control and be responsive to tasks in a wide variety of systems. In examples, machine learning systems may be used in advanced computing applications (such as online advertising, natural language processing, robotics, search engines, software engineering, speech and handwriting recognition, pattern matching, game playing, computational anatomy, bioinformatics systems and the like). In an example, machine learning may also be used in financial and marketing systems (such as for user behavior analytics, online advertising, economic estimations, financial market analysis, and the like).

Features of the system and method of the present disclosure may be implemented on both gaming machines and personal computing devices or other devices which are not specially configured for the provision of a wagering game and therefore may lack components typically included in gaming machines. Accordingly, a gaming system as used herein refers to any and all of the foregoing machines and devices, including various configurations that may include one or more central servers, central controllers, or remote hosts, one or more electronic gaming machines and/or one or more devices which are not specially configured for the provision of a wagering game, such as desktop computers, laptop computers, tablet computers or computing devices, televisions, personal digital assistants (PDAs), mobile telephones such as smart phones, and other mobile computing devices, all of which are collectively referred to by the term personal computing devices used herein.

Thus, in various embodiments, the gaming system of the present disclosure may include: one or more electronic gaming machines in combination with one or more central servers, central controllers, or remote hosts; one or more personal computing devices in combination with one or more central servers, central controllers, or remote hosts; one or more personal computing devices in combination with one or more electronic gaming machines; one or more personal computing devices, one or more electronic gaming machines, and one or more central servers, central controllers, or remote hosts in combination with one another; a single electronic gaming machine; a plurality of electronic gaming machines in combination with one another; a single personal computing device; a plurality of personal computing devices in combination with one another; a single central server, central controller, or remote host; and/or a plurality of central servers, central controllers, or remote hosts in combination with one another. In the various embodiments, the personal computing devices and/or electronic gaming machines are configured to communicate with one another and/or the central server, central controller or remote host through a communication link, such as a local or wide area data network, closed, intranet or open system or remote link such as the Internet.

For brevity and clarity, each gaming system, that is, electronic gaming machines and personal computing devices connected to a server or mentioned herein and any equivalents thereto will be collectively referred to by the term “EGM.” Additionally, for brevity and clarity, unless specifically stated otherwise, an EGM as used herein represents one EGM or a plurality of EGMs, and a central server, central controller, or remote host as used herein represents one central server, central controller, or remote host or a plurality of central servers, central controllers, or remote hosts.

In certain embodiments in which the gaming system includes an EGM in combination with a central server, central controller, or remote host, the central server, central controller, or remote host is any suitable computing device (such as a server) that includes at least one processor and at least one memory device or storage device. As further described below, the EGM includes at least one EGM processor configured to transmit and receive data or signals representing events, messages, commands, or any other suitable information between the EGM and the central server, central controller, or remote host. The at least one processor of that EGM is configured to execute the events, messages, or commands represented by such data or signals in conjunction with the operation of the EGM. Moreover, the at least one processor of the central server, central controller, or remote host is configured to transmit and receive data or signals representing events, messages, commands, or any other suitable information between the central server, central controller, or remote host and the EGM. The at least one processor of the central server, central controller, or remote host is configured to execute the events, messages, or commands represented by such data or signals in conjunction with the operation of the central server, central controller, or remote host. It should be appreciated that one, more, or each of the functions of the central server, central controller, or remote host may be performed by the at least one processor of the EGM. It should be further appreciated that one, more, or each of the functions of the at least one processor of the EGM may be performed by the at least one processor of the central server, central controller, or remote host.

In certain such embodiments, computerized instructions for controlling any games (such as any primary or base games, any secondary or bonus games and/or any non-primary and non-secondary games) displayed by the EGM are executed by the central server, central controller, or remote host. In such “thin client” embodiments, the central server, central controller, or remote host remotely controls any games (or other suitable interfaces) displayed by the EGM, and the EGM is utilized to display such games (or suitable interfaces) and to receive one or more inputs or commands. In other such embodiments, computerized instructions for controlling any games displayed by the EGM are communicated from the central server, central controller, or remote host to the EGM and are stored in at least one memory device of the EGM. In such “thick client” embodiments, the at least one processor of the EGM executes the computerized instructions to control any games (or other suitable interfaces) displayed by the EGM.

In various embodiments in which the gaming system includes a plurality of EGMs, one or more of the EGMs are thin client EGMs and one or more of the EGMs are thick client EGMs. In other embodiments in which the gaming system includes one or more EGMs, certain functions of one or more of the EGMs are implemented in a thin client environment, and certain other functions of one or more of the EGMs are implemented in a thick client environment. In one such embodiment in which the gaming system includes an EGM and a central server, central controller, or remote host, computerized instructions for controlling any primary or base games displayed by the EGM are communicated from the central server, central controller, or remote host to the EGM in a thick client configuration, and computerized instructions for controlling any secondary or bonus games or other functions displayed by the EGM are executed by the central server, central controller, or remote host in a thin client configuration.

In certain embodiments in which the gaming system includes: an EGM configured to communicate with a central server, central controller, or remote host through a data network; and/or a plurality of EGMs configured to communicate with one another through a data network, the data network is a local area network (LAN) in which the EGMs are located substantially proximate to one another and/or the central server, central controller, or remote host. In one example, the EGMs and the central server, central controller, or remote host are located in a gaming establishment or a portion of a gaming establishment.

In other embodiments in which the gaming system includes: an EGM configured to communicate with a central server, central controller, or remote host through a data network; and/or a plurality of EGMs configured to communicate with one another through a data network, the data network is a wide area network (WAN) in which one or more of the EGMs are not necessarily located substantially proximate to another one of the EGMs and/or the central server, central controller, or remote host. For example, one or more of the EGMs are located: in an area of a gaming establishment different from an area of the gaming establishment in which the central server, central controller, or remote host is located; or in a gaming establishment different from the gaming establishment in which the central server, central controller, or remote host is located. In another example, the central server, central controller, or remote host is not located within a gaming establishment in which the EGMs are located. It should be appreciated that in certain embodiments in which the data network is a WAN, the gaming system includes a central server, central controller, or remote host and an EGM each located in a different gaming establishment in a same geographic area, such as a same city or a same state. It should be appreciated that gaming systems in which the data network is a WAN are substantially identical to gaming systems in which the data network is a LAN, though the quantity of EGMs in such gaming systems may vary relative to one another.

In further embodiments in which the gaming system includes: an EGM configured to communicate with a central server, central controller, or remote host through a data network; and/or a plurality of EGMs configured to communicate with one another through a data network, the data network is an internet or an intranet. In certain such embodiments, an internet browser of the EGM is usable to access an internet game page from any location where an internet connection is available. In one such embodiment, after the internet game page is accessed, the central server, central controller, or remote host identifies a player prior to enabling that player to place any wagers on any plays of any wagering games. In one example, the central server, central controller, or remote host identifies the player by requiring a player account of the player to be logged into via an input of a unique username and password combination assigned to the player. It should be appreciated, however, that the central server, central controller, or remote host may identify the player in any other suitable manner, such as by validating a player tracking identification number associated with the player; by reading a player tracking card or other smart card inserted into a card reader (as described below); by validating a unique player identification number associated with the player by the central server, central controller, or remote host; or by identifying the EGM, such as by identifying the MAC address or the IP address of the internet facilitator. In various embodiments, once the central server, central controller, or remote host identifies the player, the central server, central controller, or remote host enables placement of one or more wagers on one or more plays of one or more primary or base games and/or one or more secondary or bonus games, and displays those plays via the internet browser of the EGM.

It should be appreciated that the central server, central controller, or remote host and the EGM are configured to connect to the communication link, data network or remote communications link in any suitable manner. In various embodiments, such a connection is accomplished via: a conventional phone line or other data transmission line, a digital subscriber line (DSL), a T-1 line, a coaxial cable, a fiber optic cable, a wireless or wired routing device, a mobile communications network connection (such as a cellular network or mobile internet network), or any other suitable medium. It should be appreciated that the expansion in the quantity of computing devices and the quantity and speed of internet connections in recent years increases opportunities for players to use a variety of EGMs to play games from an ever-increasing quantity of remote sites. It should also be appreciated that the enhanced bandwidth of digital wireless communications may render such technology suitable for some or all communications, particularly if such communications are encrypted. Higher data transmission speeds may be useful for enhancing the sophistication and response of the display and interaction with players.

In various embodiments, an EGM includes at least one processor configured to operate with at least one memory device, at least one input device, and at least one output device. The at least one processor may be any suitable processing device or set of processing devices, such as a microprocessor, a microcontroller-based platform, a suitable integrated circuit, or one or more application-specific integrated circuits (ASICs).

As generally noted above, the at least one processor of the EGM is configured to communicate with, configured to access, and configured to exchange signals with at least one memory device or data storage device. In various embodiments, the at least one memory device of the EGM includes random access memory (RAM), which can include non-volatile RAM (NVRAM), magnetic RAM (MRAM), ferroelectric RAM (FeRAM), and other forms as commonly understood in the gaming industry. In other embodiments, the at least one memory device includes read only memory (ROM). In certain embodiments, the at least one memory device of the EGM includes flash memory and/or EEPROM (electrically erasable programmable read only memory). It should be appreciated that any other suitable magnetic, optical, and/or semiconductor memory may operate in conjunction with the EGM disclosed herein. In certain embodiments, the at least one processor of the EGM and the at least one memory device of the EGM both reside within a cabinet of the EGM. In other embodiments, at least one of the at least one processor of the EGM and the at least one memory device of the EGM reside outside the cabinet of the EGM

In certain embodiments, as generally described herein, the at least one memory device of the EGM stores program code and instructions executable by the at least one processor of the EGM to control the EGM. The at least one memory device of the EGM also stores other operating data, such as image data, event data, input data, random number generators (RNGs) or pseudo-RNGs, paytable data or information, and/or applicable game rules that relate to the play of one or more games on the EGM (such as primary or base games, secondary or bonus games and any non-primary and non-secondary games as described below). In various embodiments, part or all of the program code and/or the operating data described above is stored in at least one detachable or removable memory device including, but not limited to, a cartridge, a disk, a CD ROM, a DVD, a USB memory device, or any other suitable non-transitory computer readable medium. In certain such embodiments, an operator (such as a gaming establishment operator) and/or a player uses such a removable memory device in an EGM to implement at least part of the present disclosure. In other embodiments, part or all of the program code and/or the operating data is downloaded to the at least one memory device of the EGM through any suitable data network described above (such as an internet or intranet).

In various embodiments, the EGM includes one or more input devices. The input devices may include any suitable device that enables an input signal to be produced and received by the at least one processor of the EGM. One input device of the EGM may be a payment device configured to communicate with the at least one processor of the EGM to fund the EGM or a player account which is capable of funding the EGM. In certain embodiments, the payment device includes one or more of: a bill acceptor into which paper money is inserted to fund the EGM; a ticket acceptor into which a ticket or a voucher is inserted to fund the EGM; a coin slot into which coins or tokens are inserted to fund the EGM; a reader or a validator for credit cards, debit cards, or credit slips into which a credit card, debit card, or credit slip is inserted to fund the EGM; a player identification card reader into which a player identification card is inserted to fund the EGM; through communication with a bank account or mobile device, such as a smartphone, or other account configured for transferring funds or cryptocurrency to the EGM upon authorization by a player; or any suitable combination thereof.

In one embodiment, the EGM includes a payment device configured to enable the EGM to be funded via an electronic funds transfer, such as a transfer of funds from a bank account. In another embodiment, the EGM includes a payment device configured to communicate with a mobile device of a player, such as a cell phone, a radio frequency identification tag, or any other suitable wired or wireless device, to retrieve relevant information associated with that player to fund the EGM. It should be appreciated that when the EGM is funded, the at least one processor determines the amount of funds entered and displays the corresponding amount on a credit display or any other suitable display.

Some portions of the disclosure are presented in terms of algorithms (e.g., as represented in flowcharts, prose descriptions, or both) and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic, or optical signals capable of being stored, transferred, combined, compared, and otherwise manipulated. It is convenient at times, principally for reasons of common usage, to refer to these signals as bits, values, elements, symbols, characters, terms, numbers, or the like. Furthermore, it is also convenient at times to refer to certain arrangements of steps requiring physical manipulations or transformation of physical quantities or representations of physical quantities as modules or code devices, without loss of generality. However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing,” “computing,” “calculating,” “determining,” “displaying,” “determining,” or the like, refer to the action and processes of a computer system, or similar electronic computing device (such as a specific computing machine), that manipulates and transforms data represented as physical (electronic) quantities within the computer system memories or registers or other such information storage, transmission or display devices.

Certain aspects of the embodiments include process steps and instructions described herein in the form of an algorithm. It should be noted that the process steps and instructions of the embodiments can be embodied in software, firmware, or hardware, and when embodied in software, could be downloaded to reside on and be operated from different platforms used by a variety of operating systems. The embodiments can also be in a computer program product, which can be executed on a computing system.

Some embodiments also relate to an apparatus for performing the operations herein. Such an apparatus may be specially constructed for the purposes, e.g., a specific computer, or it may comprise a general-purpose computer selectively activated or reconfigured by a computer program stored in the computer. Such a computer program may be stored in a computer-readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Memory can include any of the above and/or other devices that can store information/data/programs and can be a transient or non-transient medium, where a non-transient or non-transitory medium can include memory/storage that stores information for more than a minimal duration. Furthermore, the computers referred to in the specification may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient to construct more specialized apparatus to perform the method steps. The structure for a variety of these systems will appear from the description herein. In addition, the embodiments are not described with reference to any particular programming language. It will be appreciated that a variety of programming languages may be used to implement the teachings of the embodiments as described herein, and any references herein to specific languages are provided for the purposes of enablement and best mode.

Those skilled in the art will appreciate that the types of software and hardware used are not vital to the full implementation of the system and method of the present disclosure. The order of execution or performance of the operations in the embodiments of the invention illustrated and described herein is not essential, unless otherwise specified. That is, the operations described herein may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

While exemplary systems and methods, and applications of methods of the invention, have been described herein, it should also be understood that the foregoing is only illustrative of a few particular embodiments with exemplary and/or preferred features, as well as principles of the invention, and that various modifications can be made by those skilled in the art without departing from the scope and spirit of the invention. Additional information regarding exemplary embodiments of the invention is provided below.

### Computer Program

In some embodiments, the methods, systems, and media disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player component. In various further embodiments, the media player component utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the methods, systems, and media disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the methods, systems, and media disclosed herein include one or more databases, or use of the same. In view of the disclosure provided herein, those of skill in the art will recognize that many databases are suitable for storage and retrieval of player and game information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based.

In still further embodiments, a database is cloud computing-based. In other embodiments, a database is based on one or more local computer storage devices.

### GENERAL INFORMATION RELATING TO VARIOUS EMBODIMENTS

A controller, computing device, or computer, such as described herein, may include at least one or more processors or processing units and a system memory. The controller typically also includes at least some form of computer readable media. By way of example and not limitation, computer readable media may include computer storage media and communication media. Computer storage media may include volatile and nonvolatile, removable and non-removable media implemented in any method or technology that enables storage of information, such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art should be familiar with the modulated data signal, which has one or more of its characteristics set or changed in such a manner as to encode information in the signal. Combinations of any of the above are also included within the scope of computer readable media.

In some embodiments, a controller may include a processor, which as described herein, includes any programmable system including systems and microcontrollers, reduced instruction set circuits (RISC), application specific integrated circuits (ASIC), programmable logic circuits (PLC), and any other circuit or processor capable of executing the functions described herein. The above examples are exemplary only, and thus are not intended to limit in any way the definition and/or meaning of the term processor.

The order of execution or performance of the operations in the embodiments of the invention illustrated and described herein is not essential, unless otherwise specified. That is, the operations described herein may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

This written description uses examples to disclose the invention and also to enable any person skilled in the art to practice the invention, including making and using any devices or systems and performing any incorporated methods. The patentable scope of the invention is defined by the claims, and may include other examples that occur to those skilled in the art. Other aspects and features of the invention can be obtained from a study of the drawings, the disclosure, and the appended claims. The invention may be practiced otherwise than as specifically described within the scope of the appended claims. It should also be noted, that the steps and/or functions listed within the appended claims, notwithstanding the order of which steps and/or functions are listed therein, are not limited to any specific order of operation.

Those skilled in the art will readily appreciate that the systems and methods described herein may be a standalone system, gaming device, gaming machine or incorporated in an existing gaming system or machine. The gaming machine of the present disclosure may include various computer and network related software and hardware, such as programs, operating systems, memory storage devices, data input/output devices, data processors, servers with links to data communication systems, wireless or otherwise, and data transceiving terminals. It should also be understood that any method steps discussed herein, such as for example, steps involving the receiving or displaying of data, may further include or involve the transmission, receipt and processing of data through conventional hardware and/or software technology to effectuate the steps as described herein. Those skilled in the art will further appreciate that the precise types of software and hardware used are not vital to the full implementation of the methods of the invention so long as players and operators thereof are provided with useful access thereto, either through a mobile device, gaming platform, or other computing platform via a local network or global telecommunication network.

Although specific features of various embodiments of the invention may be shown in some drawings and not in others, this is for convenience only. In accordance with the principles of the invention, any feature of a drawing may be referenced and/or claimed in combination with any feature of any other drawing.

Those skilled in the art will readily appreciate that the apparatus described herein may include various computer and network related software and hardware, such as programs, operating systems, memory storage devices, data input/output devices, data processors, servers with links to data communication systems, wireless or otherwise, and data transceiving terminals. Those skilled in the art will further appreciate that the precise types of software and hardware used are not vital to the full implementation of the apparatus of the invention so long as it performs as described in at least one of the embodiments herein.

While exemplary apparatus, systems and methods of the invention have been described herein, it should also be understood that the foregoing is only illustrative of a few particular embodiments with exemplary and/or preferred features, as well as principles of the invention, and that various modifications can be made by those skilled in the art without departing from the scope and spirit of the invention. Therefore, the described embodiments should not be considered as limiting of the scope of the invention in any way Accordingly, all such modifications, and any equivalents thereto, are intended to be included within the scope of the embodiments of the present disclosure as defined in the following claims. In addition, the reference numerals in the claims are merely for convenience and are not to be read in any way as limiting.

What is claimed is:

1. A method for delivering a mental health resource to a person, comprising:
conducting a Tarot reading regarding a situation by pulling a combination of Tarot cards from a Tarot card deck;
receiving, by a computer processor, information regarding the combination of Tarot cards;
generating, by an artificial intelligence (AI) algorithm, a message constructed to provide insight or comment to the person regarding the situation; and
transmitting the message, by the computer processor, to the person.

2. The method of claim 1, further comprising:
determining, using a predictive analytics estimator, a future course of action having a likelihood of happening; and
wherein generating the message includes providing a recommendation regarding the future course of action.

3. The method of claim 1, wherein the message includes guidance regarding critical thinking skills or coping mechanisms.

4. The method of claim 1, wherein the message includes directions for obtaining a resource for help with the situation or with an identified emotional state.

5. The method of claim 1, further comprising: identifying demographic information regarding the person; and
wherein generating the message includes preparing the message in a voice or style based upon the demographic information regarding the person.

6. The method of claim 1, further comprising:
receiving, by the computer processor, information regarding a selection of a type of spread for the Tarot reading;
determining a main area of concern based upon the selection regarding the type of spread; and
wherein the main area of concern is a factor used by the AI algorithm for generating the message.

7. The method of claim 1, further comprising:
receiving, by the computer processor, information regarding a plurality of selections regarding types of spreads for the Tarot reading;
determining an indicator of the person's mental state based upon the information regarding the plurality of selections; and
using the indicator of the person's mental state, by the AI algorithm, for generating the message.

8. The method of claim 1, wherein transmitting the message includes sending an electronic mail (e-mail) message to the person.

9. The method of claim 1, further comprising:
asking questions of the person to assess a current mental state;
receiving, by the computer processor, answers to the questions, with the answers provided by the person; and
using the answers to the questions, by the AI algorithm, for generating the message.

10. The method of claim 1, further comprising using electronic health record (EHR) data regarding the person, by the AI algorithm, for generating the message.

11. The method of claim 1, further comprising:
receiving, by a social media miner, social media data from a social media account associated with the person;
identifying, using the social media data, areas of concern or indicia of a mental health condition by a social media parser including an AI program; and
using the areas of concern or the indicia of a mental health condition, by the AI algorithm, for generating the message.

12. A method for delivering a mental health resource to a person, comprising:
conducting a Tarot reading regarding a situation by pulling a combination of Tarot cards from a Tarot card deck;
receiving, by a computer processor, information regarding the combination of Tarot cards;
determining, by a first artificial intelligence (AI) algorithm, an emotional response likely to result from the combination of Tarot cards; and
generating, by a second AI algorithm, a message based upon the emotional response likely to result from the combination of Tarot cards, the message being constructed to provide insight or comment to the person regarding the situation.

13. A system for delivering a mental health resource to a person, comprising:
a user interface configured to present output data to the person and to receive input data from the person;
a computer processor configured to receive the input data regarding a situation from the person via the user interface and to cause the user interface to present the output data regarding a Tarot reading, the Tarot reading including pulling a combination of Tarot cards from a Tarot card deck;
a first artificial intelligence (AI) algorithm configured to determine an emotional response likely to result from the combination of Tarot cards;
a second AI algorithm configured to generate a message based upon the emotional response likely to result from the combination of Tarot cards, the message being constructed to provide insight or comment to the person regarding the situation.

14. The system of claim 13, wherein the user interface is provided by an electronic gaming machine (EGM).

15. The system of claim 13, wherein the user interface is provided by a personal computing device.

16. The system of claim 13, wherein at least one of the first AI algorithm or the second AI algorithm is provided as a computer program running on a server remote from the user interface and in communication therewith over a digital computer network.

17. The system of claim 13, further comprising a predictive analytics estimator configured to determine a future course of action having a likelihood of happening; and
wherein the second AI algorithm is configured to generate the message by providing a recommendation regarding the future course of action.

18. The system of claim 13, wherein the message includes guidance regarding critical thinking skills or coping mechanisms, or wherein the message includes directions for obtaining a resource for help with the situation or with an identified emotional state.

19. The system of claim 13, wherein the message is transmitted as an electronic mail (e-mail) message to the person.

20. The system of claim 13, wherein the user interface is configured to ask questions designed to assess a current mental state; and
wherein the second AI algorithm is configured to use answers to the questions for generating the message.
